(19) United States
(12) Patent Application Publication
(10) Pub. No.: US 2025/0243152 A1
(43) Pub. Date: Jul. 31, 2025

(54) INTERMEDIATE OF POLYAMINE DERIVATIVE PHARMACEUTICAL SALT, PREPARATION METHOD THEREFOR, AND USE THEREOF

(71) Applicant: WUHAN WUYAO SCI & TECH CO., LTD., Wuhan, Hubei (CN)

(72) Inventors: Xiang RAO, Wuhan, Hubei (CN); Fei WENG, Wuhan, Hubei (CN); Yishun XIA, Wuhan , Hubei (CN); Xinrong LIU, Wuhan, Hubei (CN); Zhenwu LI, Wuhan, Hubei (CN); Bo YANG, AA (CN); Lei LI, Wuhan, Hubei (CN)

(21) Appl. No.: 18/854,881

(22) PCT Filed: Apr. 26, 2023
(86) PCT No.: PCT/CN2023/090767

§ 371 Date: Oct. 7, 2024

(30) Foreign Application Priority Data
Apr. 27, 2022 (CN) 202210452147.9

(51) Int. Cl. C07C 235/34; C07C 231/12; C07C 271/20

(52) U.S. Cl. CPC C07C 235/34; C07C 231/12; C07C 271/20

(57) ABSTRACT

Disclosed are an intermediate of a polyamine derivative pharmaceutical salt, a preparation method therefor, and use thereof. The intermediate compound has the following structure: (I), which can be used to prepare a polyamine derivative and a pharmaceutical salt thereof, and has very good application prospects in the field of chemical medicine.

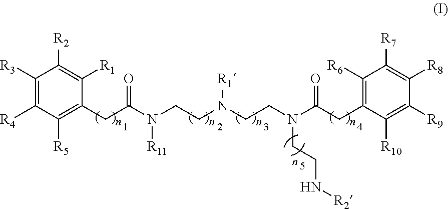

### TECHNICAL FIELD

The present invention relates to the technical field of chemical medicines, and particularly relates to an intermediate of a polyamine derivative pharmaceutical salt, a preparation method therefor, and use thereof.

### BACKGROUND

Systemic inflammatory response syndrome and autoimmune disorder-related diseases, such as sepsis and autoimmune diseases, are two categories of diseases caused by the body's own excessive immune response. Currently, there is still a lack of effective therapeutic medicaments, and the targeted prevention and treatment of these diseases are the focus and hot issues of clinical concern. Sepsis refers to systemic inflammatory response syndrome (SIRS) mediated by infectious factors, which affects up to 19 million people worldwide every year. Despite significant advances in current antibiotics and critical medicine technology, sepsis remains a major cause of death in infected patients, and there is no ideal therapeutic medicaments to date.

Studies show that the pathogenesis of sepsis lies in the fact that pathogen-associated molecular patterns (PAMPs) released by bacteria, viruses, fungi, and other pathogens are recognized by pattern recognition receptors (PRRs) of the host's innate immune system, mediating the activation of inflammatory response cells, and thus causing a systemic excessive inflammatory response. Epidemiological surveys show that PAMP molecules that cause sepsis mainly include bacterial lipopolysaccharide (LPS), bacterial genomic DNA (CpG DNA), peptidoglycan (PGN), lipoteichoic acid (LTA), viral RNA, and zymosan. However, there are currently few effective medicaments for the treatment of sepsis.

The granted Chinese invention patent with the publication No. CN105348137B discloses a polyamine derivative pharmaceutical salt, a preparation method therefor, and use thereof. The polyamine derivative pharmaceutical salt can be used in the preparation of a medicament for treating sepsis. However, in the preparation process, the purity of intermediates and final product is not considered, and the preparation method needs to be improved.

Therefore, it is very necessary to develop a preparation method suitable for industrialization that can obtain a polyamine derivative pharmaceutical salt and an intermediate thereof with high purity.

### SUMMARY

To overcome the defects in the prior art, in a first aspect of the present invention, the present invention provides a compound having the following structure:

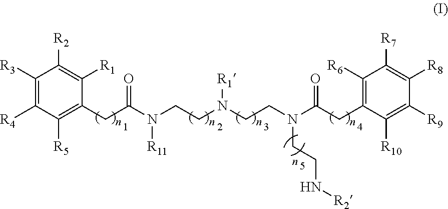

wherein,
R₁-R₁₀ are independently selected from: H, OH, alkoxy, aryloxy, and aralkoxy;
R₁₁ is R₃′ or

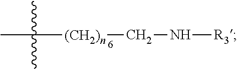

n₁-n₆ are independently selected from an integer of 0-10 (e.g., 0, 1, 2, 3, 4, 5, 6, 7, 8, 9, or 10);
R₁′ is —X₁—R₁″, wherein X₁ is C(O)O or S(O)₂, and R₁″ is selected from: alkyl, alkenyl, aralkyl, and aryl;
R₂′ is —X₂—R₂″, wherein X₂ is C(O)O or S(O)₂, and R₂″ is selected from: alkyl, alkenyl, aralkyl, and aryl; and
R₃′ is —X₃—R₃″, wherein X₃ is C(O)O or S(O)₂, and R₃″ is selected from: alkyl, alkenyl, aralkyl, and aryl.

In one specific embodiment, R₁₁ is

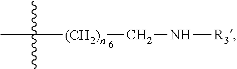

and the compound described above has the following structure:

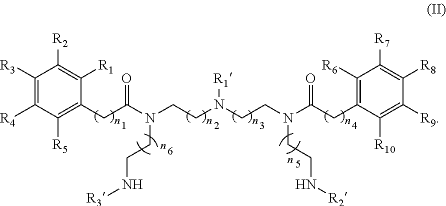

Specifically, R₁ is H.

Specifically, R₄ is H.

Specifically, R₅ is H.

Specifically, R₆ is H.

Specifically, R₉ is H.

Specifically, R₁₀ is H.

Specifically, R₂, R₃, R₇, and R₈ are independently selected from: H, OH, alkoxy, aryloxy, and aralkoxy; more specifically, R₂, R₃, R₇, and R₈ are independently selected from: H, OH, C₁-C₆ alkoxy, C₆-C₁₂ aryloxy, and C₇-C₁₂ aralkoxy; further specifically, R₂, R₃, R₇, and R₈ are independently selected from: H, OH, and C₁-C₆ alkoxy; and furthermore specifically, R₂, R₃, R₇, and R₈ are independently selected from: OH, methoxy, and ethoxy.

Specifically, R₂ and R₃ are identical, and/or R₇ and R₈ are identical; preferably, R₂, R₃, R₇, and R₈ are all identical.

Specifically, n₁ is an integer of 1-5, for example, an integer of 1-3, for example, 2.

Specifically, n₂ is an integer of 0-5, for example, an integer of 1-3, for example, 2.

Specifically, n₃ is an integer of 0-5, for example, an integer of 1-3, for example, 2.

Specifically, n₄ is an integer of 1-5, for example, an integer of 1-3, for example, 2.

Specifically, n₅ is an integer of 0-5, for example, an integer of 1-3, for example, 2.

Specifically, n₆ is an integer of 0-5, for example, an integer of 1-3, for example, 2.

Specifically, R₁″, R₂″, and R₃″ are independently selected from: C₁-C₆ alkyl, C₂-C₆ alkenyl, C₆-C₁₂ aryl, and C₇-C₁₂ aralkyl; and particularly, R₁″′, R₂′″, and R₃′″ are independently selected from C₁-C₆ alkyl.

More specifically, R₁″′, R₂″, and R₃″ are independently selected from: methyl, ethyl, tert-butyl, p-methylphenyl, allyl, and fluorenylmethyl; particularly, R₁″, R₂″, and R₃″′ are independently selected from: methyl, ethyl, and tert-butyl.

Specifically, R₁′, R₂′, and R₃′ are independently selected from: —C(O)O(C₁-C₆ alkyl), —C(O)O(C₂-C₆ alkenyl), —C(O)O(C₆-C₁₂ aryl), and —C(O)O(C₇-C₁₂ aralkyl); particularly, R₁′, R₂′, and R₃′ are independently selected from —C(O)O(C₁-C₆ alkyl).

More specifically, R₁′, R₂′, and R₃′ are independently selected from: tert-butoxycarbonyl, ethoxycarbonyl, methoxycarbonyl, allyloxycarbonyl, fluorenylmethoxycarbonyl, p-toluenesulfonyl, and mesyl; particularly, R₁′, R₂′, and R₃′ are independently selected from: tert-butoxycarbonyl, ethoxycarbonyl, and methoxycarbonyl.

Specifically, R₂′ and R₃′ are identical; particularly, R₁′, R₂′, and R₃′ are identical. More specifically, R₂′ and R₃′ are identical and are selected from —C(O)O(C₁-C₆ alkyl), and specifically selected from: tert-butoxycarbonyl, ethoxycarbonyl, and methoxycarbonyl. Further specifically, R₁′, R₂′, and R₃′ are identical and are selected from —C(O)O(C₁-C₆ alkyl), and specifically selected from: tert-butoxycarbonyl, ethoxycarbonyl, and methoxycarbonyl.

In another specific embodiment, the compound described above has the following structure:

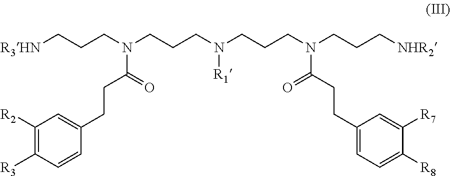

wherein R₁′, R₂′, R₃′, R₂, R₃, R₇, and R₈ have the corresponding definitions described above of the present invention.

In yet another specific embodiment, the compound described above has the following structure:

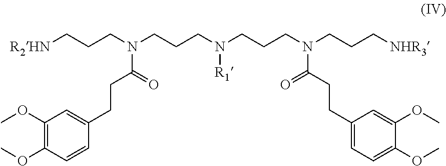

wherein R₁′, R₂′, and R₃′ have the corresponding definitions described above of the present invention.

In a second aspect of the present invention, the present invention further provides a preparation method for the compound described above, wherein the compound is prepared from a compound of formula V, and the compound of formula V has a structural formula shown as follows:

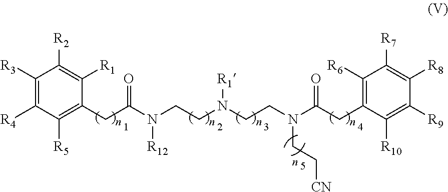

wherein R₁₂ is R₃′ or

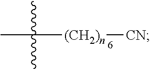

R₁-R₁₀, n₁-n₆, R₁′, and R₃′ have the corresponding definitions described above of the present invention.

In one specific embodiment, the preparation method of the present invention comprises a step of reacting a compound of formula V with reagents R₁₃-R₂′ and/or R₁₃-R₃′, wherein R₁₃ is a leaving group, for example, —O—R₂′ or —O—R₃′, wherein R₂′ and R₃′ have the corresponding definitions described above of the present invention.

Specifically, the reaction system further comprises a solvent, such as, an alcohol, such as ethanol, isopropanol, or tert-butanol, particularly ethanol.

Specifically, the reaction system further comprises a catalyst, such as Raney Ni or palladium on carbon.

In another specific embodiment, the preparation method of the present invention comprises mixing the compound of formula V with reagents R₁₃-R₂′ and/or R₁₃-R₃′, alcohol, and a catalyst, and reacting.

Specifically, the reagents are R₂′—O—R₂′ and/or R₃′—O—R₃′, wherein R₂′ and R₃′ have the corresponding definitions described above of the present invention, such as

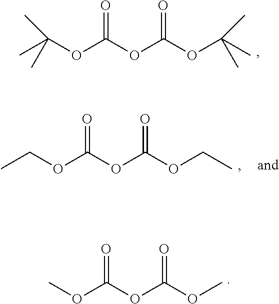

In some embodiments of the present invention, R₂′ and R₃′ are identical; particularly, R₁′, R₂′, and R₃′ are identical.

In the preparation method of the present invention, specifically, the reaction is performed at a temperature of 40-50° C., such as 40° C., 42° C., 44° C., 45° C., 46° C., 48° C., or 50° C., particularly, 45° C.

In the preparation method of the present invention, specifically, the reaction is performed under pressure of 1.0-2.0 MPa.

In the preparation method of the present invention, specifically, the reaction is performed under stirring.

In the preparation method of the present invention, specifically, the reaction is performed for a period of 24-72 h, for example, 24 h, 35 h, 48 h, 60 h, and 72 h.

In yet another specific embodiment, the preparation method of the present invention further comprises a purification step, for example, by column chromatography.

Specifically, an eluent for the column chromatography is a mixture of acetone and n-heptane, specifically acetone:n-heptane=1:2 (v/v).

In a third aspect of the present invention, the present invention further provides use of the compound of formula I and a preparation method therefor in the preparation of a polyamine derivative (shown in formula (VI) below) and a pharmaceutically acceptable salt thereof.

In a fourth aspect of the present invention, the present invention further provides use of the compound of formula I and a preparation method therefor in the preparation of an anti-PAMP medicament.

Specifically, the PAMP is selected from: one or more of bacterial lipopolysaccharide (LPS), bacterial genomic DNA (CpG DNA), peptidoglycan (PGN), lipoteichoic acid (LTA), viral RNA, and zymosan.

In a fifth aspect of the present invention, the present invention further provides use of the compound of formula I and a preparation method therefor in the preparation of a medicament for preventing and/or treating systemic inflammatory response syndrome (SIRS).

Specifically, the systemic inflammatory response syndrome is sepsis.

In a sixth aspect of the present invention, the present invention further provides use of the compound of formula I and a preparation method therefor in preparation of a medicament for preventing and/or treating an autoimmune disease.

Specifically, the autoimmune disease is selected from: one or more of organ-specific autoimmune disease, systemic lupus erythematosus, rheumatoid arthritis, systemic vasculitis, scleroderma, pemphigus, dermatomyositis, mixed connective tissue disease, autoimmune hemolytic anemia, thyroid autoimmune disease, and ulcerative colitis.

In a seventh aspect of the present invention, the present invention further provides a preparation method for a polyamine derivative having the following structure:

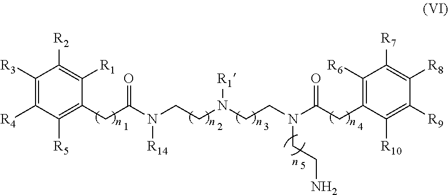

wherein R₁₄ is H or

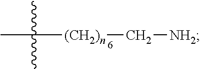

R₁-R₁₀, n₁-n₆, and R₁′ have the corresponding definitions described above of the present invention.

The preparation method comprises a step of using the compound of formula (I) described above of the present invention.

In one specific embodiment, the preparation method of the present invention comprises mixing the compound of formula (I) with a solvent, slowly adding a removal reagent, and reacting. Specifically, in the compound of formula (I), R₁′, R₂′, and R₃′ are independently selected from —C(O)OC₁-C₆ alkyl; more specifically, R₁′, R₂′, and R₃′ are independently selected from: tert-butoxycarbonyl, ethoxycarbonyl, and methoxycarbonyl.

Specifically, the removal reagent is a solution of hydrogen chloride in an organic solvent; the organic solvent may be selected from: one or more of ethyl acetate, cyclopentyl methyl ether, isopropyl acetate, methyl tert-butyl ether, methyl acetate, and propyl acetate.

Specifically, the solvent is dichloromethane.

Specifically, the reaction is performed at a temperature of 0-25° C., such as 1° C., 2° C., 3° C., 4° C., 5° C., 6° C., 7° C., 8° C., 9° C., 10° C., 15° C., 20° C., or 25° C., preferably 0-10° C., more preferably 0-5° C.

Specifically, the reaction is performed under stirring.

Specifically, the reaction is performed for a period of 1-6 h, for example, 1 h, 2 h, 3 h, 4 h, 5 h, or 6 h.

In another specific embodiment, the preparation method of the present invention further comprises a step of quenching the reaction, for example, quenching the reaction by adding water to the reaction system.

In yet another specific embodiment, the preparation method of the present invention further comprises a purification step.

Specifically, the purification step described above comprises subjecting the quenched reaction system to liquid separation, taking an aqueous phase, adjusting the pH of the aqueous phase to alkaline (e.g., pH 12-14), adding an extracting agent for extraction, combining organic phases, drying, and concentrating under reduced pressure; specifically, the extracting agent is dichloromethane.

In yet still another specific embodiment, the preparation method of the present invention further comprises the preparation of the compound of formula (I), for example, the preparation method for the compound of formula (I) described above.

Specifically, the preparation method for the polyamine derivative described above comprises the following reaction scheme:

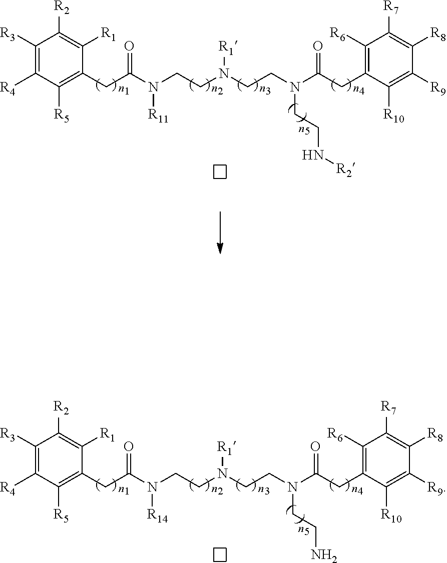

More specifically, the preparation method for the polyamine derivative described above comprises the following reaction scheme:

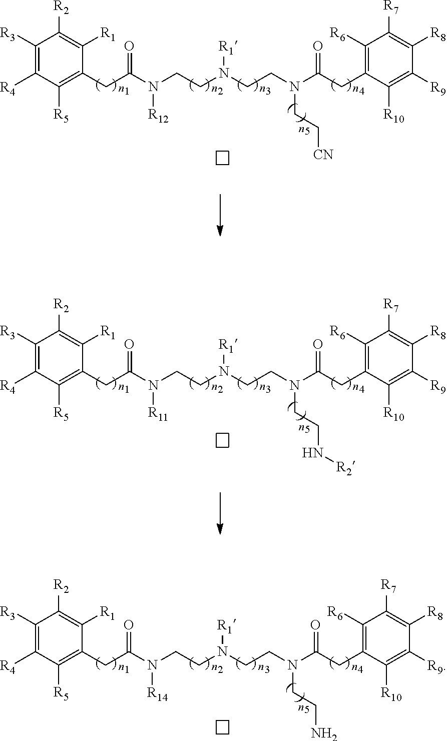

In one specific embodiment of the present invention, the preparation method for the polyamine derivative described above comprises the following steps:
(1) mixing a compound of formula (V) with reagents R₁₃-R₂′ and/or R₁₃-R₃′, alcohol, and a catalyst, and reacting;
(2) mixing the product obtained in step (1) with a solvent, slowly adding a removal reagent, and reacting;
(3) quenching the reaction in step (2); and
(4) subjecting the reaction system quenched in step (3) to liquid separation, taking an aqueous phase, adjusting the pH of the aqueous phase to alkaline, adding an extracting agent for extraction, combining organic phases, drying, and concentrating under reduced pressure.

Specifically, the reagents, solvents, catalysts, and reaction conditions in the preparation method described above have the corresponding definitions described above of the present invention.

In an eighth aspect of the present invention, the present invention further provides a preparation method for a pharmaceutically acceptable salt of a polyamine derivative, which comprises a step of using the polyamine derivative described above of the present invention.

In one specific embodiment, the preparation method for the pharmaceutically acceptable salt of the polyamine derivative of the present invention comprises the steps of the preparation method for the polyamine derivative described above of the present invention.

In another specific embodiment, the preparation method for the pharmaceutically acceptable salt of the polyamine derivative of the present invention further comprises a salt formation step by reacting the polyamine derivative with an acid.

Specifically, the salt formation step comprises slowly adding a solution of an acid in organic solvent to the solution of the polyamine derivative and performing a reaction.

Specifically, the acid is a pharmaceutically acceptable acid, including inorganic acids such as hydrochloric acid, sulfuric acid, phosphoric acid, and nitric acid, and organic acids such as acetic acid, oxalic acid, malonic acid, succinic acid, benzoic acid, trifluoroacetic acid, maleic acid, fumaric acid, citric acid, tartaric acid, methanesulfonic acid, benzenesulfonic acid, and p-toluene sulfonic acid.

Specifically, the organic solvent is ethyl acetate.

Specifically, the step of slowly adding a solution of an acid in an organic solvent described above is performed at a temperature of 0-15° C., such as 0° C., 1° C., 2° C., 3° C., 4° C., 5° C., 6° C., 7° C., 8° C., 9° C., 10° C., 11° C., 12° C., 13° C., 14° C., or 15° C., particularly, 0-10° C.

Specifically, the reaction is performed under stirring.

Specifically, the reaction is performed for a period of 1-6 h, for example, 1 h, 2 h, 3 h, 4 h, 5 h, or 6 h.

In yet another specific embodiment, the preparation method for the pharmaceutically acceptable salt of the polyamine derivative of the present invention further comprises a purification step.

In yet still another specific embodiment of the present invention, the preparation method for the pharmaceutically acceptable salt of the polyamine derivative comprises the following steps:
(1) mixing a compound of formula (V) with reagents R₁₃-R₂′ and/or R₁₃-R₃′, alcohol, and a catalyst, and reacting;
(2) mixing the product obtained in step (1) with a solvent, slowly adding a removal reagent, and reacting;
(3) quenching the reaction in step (2);
(4) subjecting the reaction system quenched in step (3) to liquid separation, taking an aqueous phase, adjusting the pH of the aqueous phase to alkaline, adding an extracting agent for extraction, combining organic phases, drying, and concentrating under reduced pressure; and
(5) slowly adding a solution of an acid in an organic solvent to the product obtained in step (4), and reacting.

Specifically, the reagents, solvents, catalysts, and reaction conditions in the preparation method described above have the corresponding definitions described above of the present invention.

The present invention provides a compound that can be used as an intermediate in the preparation of a polyamine derivative and a pharmaceutically acceptable salt thereof. The compound is easy to prepare and purify. When the compound is used in the preparation of the polyamine derivative and the pharmaceutically acceptable salt thereof, the yield and the purity of the obtained product can be significantly improved, and the operation is simple, convenient, and quick, thereby facilitating the improvement of the industrial production of the polyamine derivative and the pharmaceutically acceptable salt thereof. The compound has very good application prospects in the field of chemical medicine. The compound described above that can be used as an intermediate in the preparation of a polyamine derivative and a pharmaceutically acceptable salt thereof is obtained by the inventors through extensive experimental screening. Although various groups can be used for protecting amino in the prior art, the inventors have found through experiments that, in the main structure of the compound of the present invention, some protecting groups (e.g., triphenylmethyl (Trt), benzyl (Bn), etc.) are not ideal for use due to various reasons such as easy poisoning and deactivation of catalysts in the protection reaction, easy removal of the protective groups after preparation, poor stability in the process, etc.

### BRIEF DESCRIPTION OF THE DRAWINGS

FIG. 1 shows the results of nuclear magnetic resonance hydrogen spectrum for compound 2a. Instrument model: Bruker avance 400 (400 MHz) nuclear magnetic resonance spectrometer; test condition: 400 MHz; solvent: deuterated chloroform.

FIG. 2 shows the results of mass spectrum for compound 2a. Instrument model: Agilent Technologies 6530 Accurate-Mass Q-TOF LC/MS; test condition: ESI, fragmentor voltage: 175V, collision energy: 0.

### DETAILED DESCRIPTION

Unless otherwise defined, all scientific and technical terms used in the present invention have the same meaning as commonly understood by those skilled in the art to which the present invention relates.

The term “alkyl” refers to linear or branched hydrocarbyl that does not contain unsaturated bonds and that is attached to the rest of the molecule via a single bond. The alkyl as used herein generally contains 1 to 12 (e.g., 1, 2, 3, 4, 5, 6, 7, 8, 9, 10, 11, or 12) carbon atoms, preferably 1 to 6 carbon atoms (i.e., C₁-C₆ alkyl). Examples of alkyl include, but are not limited to, methyl, ethyl, n-propyl, isopropyl, n-butyl, isobutyl, tert-butyl, n-pentyl, isopentyl, neopentyl, tert-pentyl, n-hexyl, isohexyl, and the like. If alkyl is substituted with aryl, it is correspondingly “aralkyl” (e.g., C₇-C₁₈ aralkyl, e.g., C₇-C₁₅ aralkyl or C₇-C₁₂ aralkyl; e.g., (C₁-C₆ alkylene)-(C₆-C₁₂ aryl) or (C₁-C₃ alkylene)-phenyl), such as benzyl, benzhydryl, or phenethyl.

The term “alkenyl” refers to linear or branched hydrocarbyl that contains at least two carbon atoms and at least one unsaturated bond and that is attached to the rest of the molecule via a single bond. Alkenyl as used herein generally contains 2 to 12 (e.g., 2, 3, 4, 5, 6, 7, 8, 9, 10, 11, or 12) carbon atoms, preferably 2 to 6 carbon atoms (i.e., C₂-C₆ alkenyl). Examples of alkenyl include, but are not limited to, ethenyl, 1-methyl-ethenyl, 1-propenyl, 2-propenyl, 3-propenyl (also referred to as allyl), butenyl, or the like.

The term “alkoxy” refers to a substituent formed from hydroxyl by substituting the hydrogen atom with alkyl. The alkoxy as used herein generally contains 1 to 12 (e.g., 1, 2, 3, 4, 5, 6, 7, 8, 9, 10, 11, or 12) carbon atoms, preferably 1 to 6 carbon atoms (i.e., C₁-C₆ alkoxy). Examples of alkoxy include, but are not limited to, methoxy, ethoxy, propoxy, butoxy, and the like. A substituent formed from hydroxyl by substituting the hydrogen atom with aryl is aryloxy, and the aryloxy used herein generally contains 6 to 18 (e.g., 6, 8, 10, 12, 14, 16, or 18) carbon atoms, preferably 6 to 12 carbon atoms (i.e., C₆-C₁₂ aryloxy). Examples of aryloxy include, but are not limited to, phenoxy. A substituent formed from hydroxyl in alkoxy by substituting the hydrogen atom with aralkyl is aralkoxy, and the aralkoxy used herein generally contains 7 to 18 (e.g., 7, 8, 9, 10, 11, 12, 13, 14, 15, 16, 17, or 18) carbon atoms, preferably 7 to 12 carbon atoms (i.e., C₇-C₁₂ aralkoxy). Examples of aralkoxy include, but are not limited to, benzyloxy.

The term “aryl” refers to a functional group or substituent derived from a simple aromatic ring, including monocyclic aryl groups and/or fused aryl groups, such as monocyclic or bicyclic aryl groups containing 1-3 rings and having 6-18 (e.g., 6, 8, 10, 12, 14, 16, or 18) carbon ring atoms. Aryl as used herein is generally monocyclic or bicyclic aryl containing 1-2 rings and having 6-12 carbon ring atoms (i.e., C₆₋₁₂ aryl), wherein H on the carbon atoms may be substituted, for example, with alkyl, halogen, and other groups. Examples of aryl include, but are not limited to, phenyl, p-methylphenyl, naphthyl, biphenyl, indenyl, and the like.

The term “halogen” refers to bromine, chlorine, iodine, or fluorine.

The term “systemic inflammatory response syndrome” or “SIRS” refers to a response related to at least two of the following criteria: temperature>38° C. or <36° C., heart rate >90/min, respiratory rate>20/min or paCO₂<32 milliliters of mercury (mmHg), leukocyte count>12×10⁹/L or <4×10⁹/L, or immature leukocyte more than 10% (Bone R C; Balk R A; Cerra F B., et al., Definitions for sepsis and organ failure and guidelines for the use of innovative therapies in sepsis. The ACCP/SCCM Consensus Conference Committee. American College of Chest Physicians/Society of Critical Care Medicine[J]. Chest, 1992, 101(6):1644-1655.). SIRS may be caused by infection or trauma of any other type, particularly of the burn, surgery, or injury type. Sepsis, severe sepsis, and septic shock all correspond to SIRS caused by infection. In a patient in the state of sepsis (sepsis, severe sepsis, and septic shock), who presents SIRS as a result of infection, the infection that causes SIRS may arise from a number of sources, particularly from bacterial, viral, or fungal sources.

The term “autoimmune disease” refers to a disorder resulting from an autoimmune response. An autoimmune disease is the result of inappropriate and excessive responses to self-antigens. Autoimmune diseases include, but are not limited to: one or more of organ-specific autoimmune disease, systemic lupus erythematosus, rheumatoid arthritis, systemic vasculitis, scleroderma, pemphigus, dermatomyositis, mixed connective tissue disease, autoimmune hemolytic anemia, thyroid autoimmune disease, and ulcerative colitis.

The disclosures of the various publications, patents, and published patent specifications cited herein are hereby incorporated by reference in their entireties.

The technical solutions of the present invention will be clearly and completely described below with reference to the examples of the present invention, and it is obvious that the described examples are only a part of the examples of the present invention but not all of them. Based on the examples of the present invention, all other examples obtained by those of ordinary skills in the art without creative work shall fall within the protection scope of the present invention.

### PREPARATION AND PURIFICATION OF COMPOUNDS

### Example 1

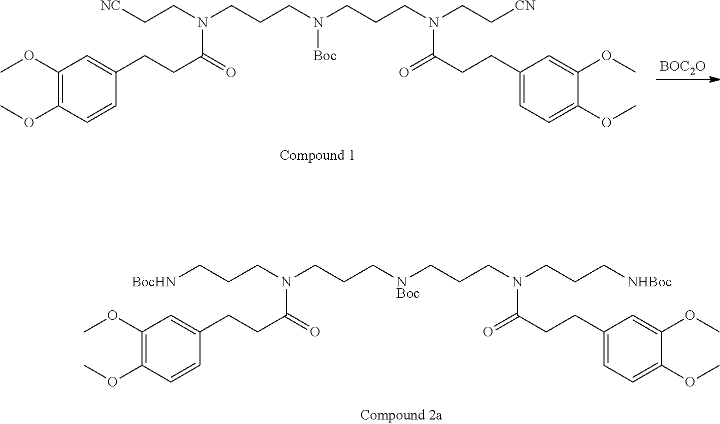

Compound 1, di-tert-butyl dicarbonate (Boc₂O or BOC₂O), ethanol, and Raney Ni were added to an autoclave. The mixture was reacted under stirring at 45° C., 1.0-2.0 MPa for 48 h. The mixture was concentrated to dryness under reduced pressure to give a crude product of Compound 2a (72.2% purity, 100.8% yield).

The crude product of Compound 2a was purified by column chromatography (200-300 mesh silica gel chromatographic column), and eluted with acetone:n-heptane=1:2 (v/v) to give Compound 2a (96.2% purity, 56.4% overall yield).

### Example 2

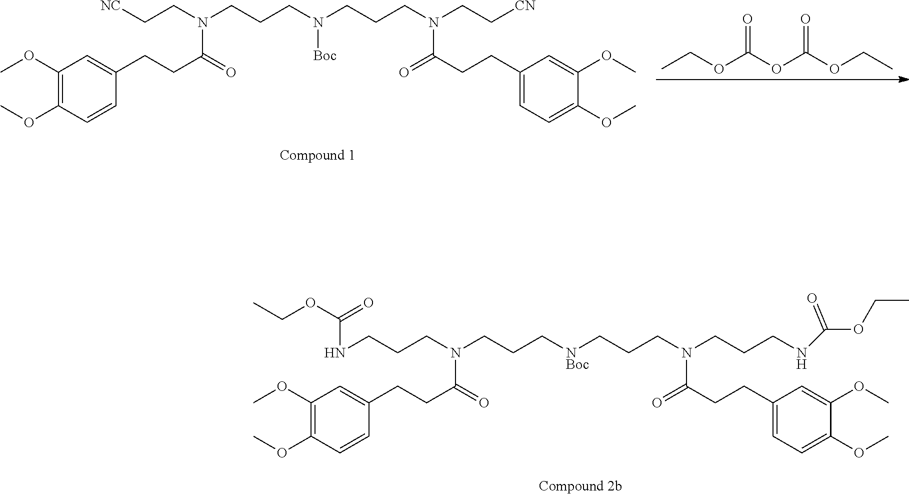

Compound 1, diethyl pyrocarbonate, isopropanol, and Raney Ni were added to an autoclave. The mixture was reacted under stirring at 40° C., 1.0-2.0 MPa for 48 h. The mixture was concentrated to dryness under reduced pressure to give a crude product of Compound 2b (69.8% purity, 99.4% yield).

The crude product of Compound 2b was purified by column chromatography (200-300 mesh silica gel chromatographic column), and eluted with acetone:n-heptane=1:2 (v/v) to give Compound 2b (93.8% purity, 55.1% overall yield).

### Example 3

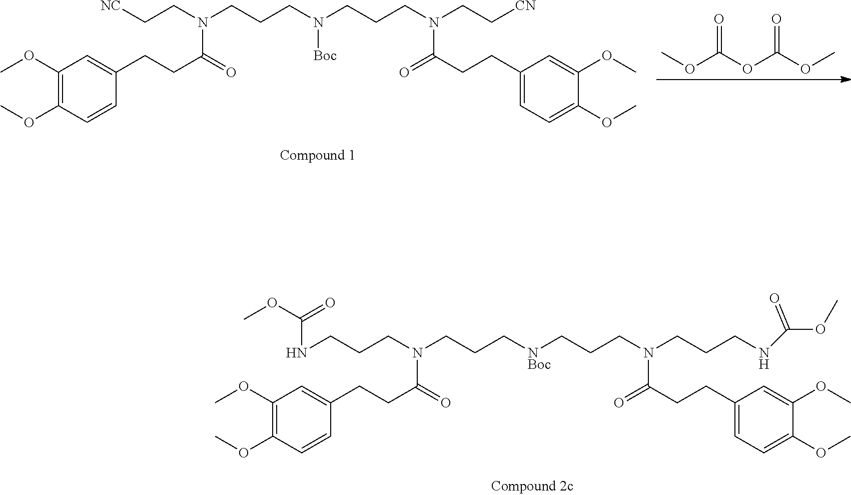

Compound 1, diethyl pyrocarbonate, tert-butanol, and Raney Ni were added to an autoclave. The mixture was reacted under stirring at 50° C., 1.0-2.0 MPa for 48 h. The mixture was concentrated to dryness under reduced pressure to give a crude product of Compound 2c (68.7% purity, 100.2% yield).

The crude product of Compound 2c was purified by column chromatography (200-300 mesh silica gel chromatographic column), and eluted with acetone:n-heptane=1:2 (v/v) to give Compound 2c (91.1% purity, 53.5% overall yield).

### Example 4

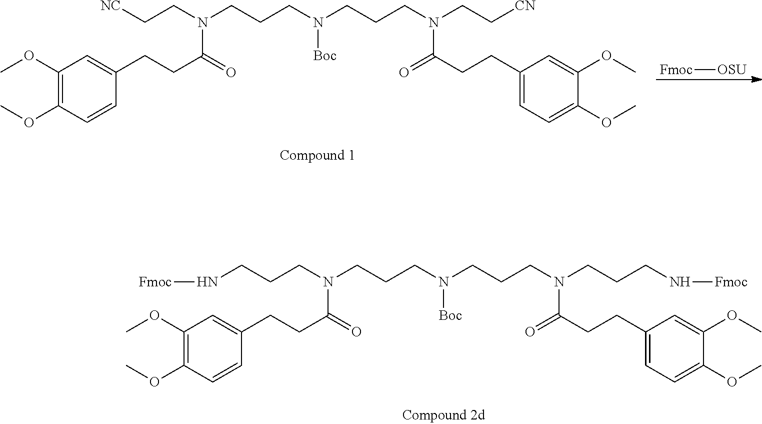

Compound 1, N-(9-fluorenylmethoxycarbonyloxy) succinimide (Fmoc-OSU), ethanol, and Raney Ni were added to an autoclave. The mixture was reacted under stirring at 45° C., 1.0-2.0 MPa for 48 h. The mixture was concentrated to dryness under reduced pressure to give a crude product of Compound 2d (16.8% purity, 39.0% yield).

### Comparative Example 1

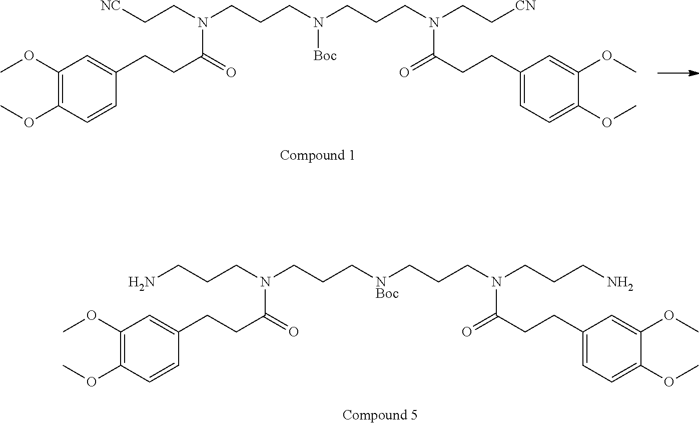

Compound 1, ethanol, and Raney Ni were added to an autoclave. The mixture was reacted under stirring at 45° C., 1.0-2.0 MPa for 48 h. The mixture was concentrated to dryness under reduced pressure to give a crude product of Compound 5 (69.2% purity, 65.9% yield).

The first purification method: the crude product of Compound 5 was purified by column chromatography (200-300 mesh silica gel chromatographic column), and eluted with acetone:n-heptane=4:1 (v/v) to give Compound 5 (72.4% purity, 30.5% overall yield). After trying purification by column chromatography, the purity and yield were still very low.

The second purification method: the crude product of Compound 5 was added to dichloromethane (DCM). The mixture was stirred until the solid was dissolved to give a clear solution. With the temperature controlled at 0-10° C., the solution was extracted with 1 M hydrochloric acid twice. The aqueous phases were combined, adjusted with 1 M sodium hydroxide to above pH 10, and extracted with DCM three times. The extract was washed with 10% sodium chloride twice and concentrated to dryness. The operations described above were repeated 3 times to give Compound 5 (96.1% purity, 6.4% yield). This purification method had a certain impurity removal effect, but there was a serious emulsification phenomenon in the alkali extraction process, and the process was complex, so that the method is not beneficial to scale-up production.

### Comparative Example 2

The reaction scheme is as shown in Comparative Example 1.

Compound 1, a saturated solution of ammonia in methanol, and Raney Ni were added to an autoclave. The mixture was reacted under stirring at 45° C., 1.0-2.0 MPa for 48 h. The mixture was concentrated to dryness under reduced pressure to give a crude product of Compound 5 (18.5% purity, 44.3% yield).

### Boc(tert-butoxycarbonyl) Removal and Salt Formation

### Example 5

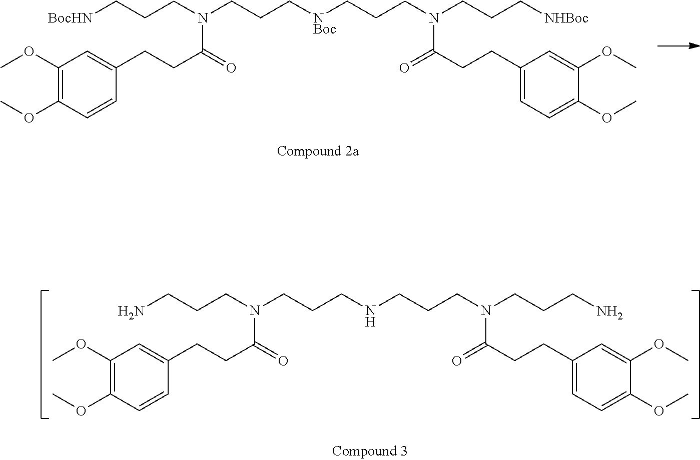

15 g of Compound 2a obtained by purification in Example 1 was added to a three-necked flask. 150.0 g of dichloromethane was added. The system was stirred until the solid was dissolved to give a clear solution. With the temperature controlled at 10° C., 78.3 g of 15% ethyl acetate hydrogen chloride (EA HCl) was slowly added. The mixture was reacted under stirring for 2 h. 75.0 g of water was added to the reaction solution, and stirred for quenching. The mixture was left to stand for liquid separation. The lower organic phase was discarded. The upper aqueous phase was adjusted to pH 12-14 with 4 M sodium hydroxide, and extracted with dichloromethane three times. The organic phases were combined, dried over anhydrous sodium sulfate, and concentrated under reduced pressure to give a solution of Compound 3 in dichloromethane with a purity of 98.2%.

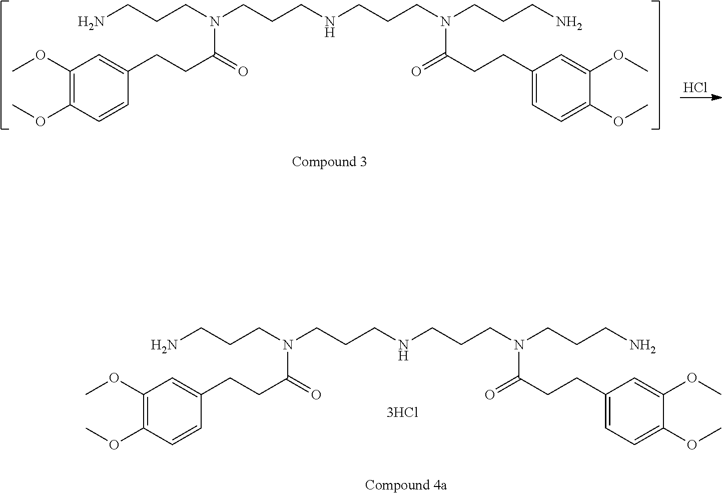

The solution of Compound 3 in dichloromethane obtained in the previous step was added to a 250 mL three-neck flask, and the solution was cooled to 0° C. 4 M ethyl acetate solution of hydrochloric acid was slowly added. The mixture was reacted under stirring for 3-5 h with the temperature controlled at 0-10° C. 30 g of methanol was added to the reaction flask with the temperature controlled at 20-30° C. The mixture was stirred and concentrated under reduced pressure at a temperature below 30° C. 60 g of ethyl acetate was added to the concentrate. The mixture was concentrated under reduced pressure at a temperature below 30° C. The filter cake was rinsed with ethyl acetate, and dried under vacuum to give an off-white solid with a yield of 85.3% and a purity of 92.5%.

### Example 6

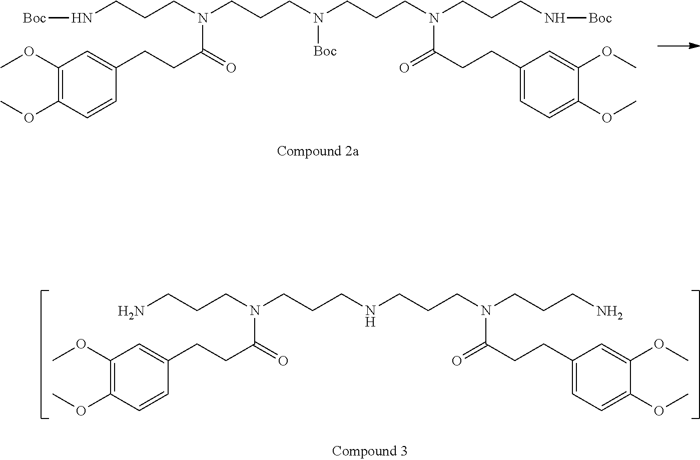

15 g of Compound 2a obtained by purification in Example 1 was added to a three-necked flask. 150.0 g of dichloromethane was added. The system was stirred until the solid was dissolved to give a clear solution. With temperature controlled at 0° C., 78.3 g of 15% cyclopentyl methyl ether hydrogen chloride (CPME·HCl) was slowly added. The mixture was reacted under stirring for 2 h. 75.0 g of water was added to the reaction solution, and stirred for quenching. The mixture was left to stand for liquid separation. The lower organic phase was discarded. The upper aqueous phase was adjusted to pH 12-14 with 4 M sodium hydroxide, and extracted with dichloromethane three times. The organic phases were combined, dried over anhydrous sodium sulfate, and concentrated under reduced pressure to give a solution of Compound 3 in dichloromethane with a purity of 98.0%.

60 g of the solution of Compound 3 in dichloromethane obtained in the previous step was added to a 250 mL three-neck flask, and the solution was cooled to 10° C. 0.5 M ethyl acetate solution of oxalic acid was slowly added. The mixture was reacted under stirring for 3-5 h with the temperature controlled at 0-10° C. 30 g of methanol was added to the reaction flask with the temperature controlled at 20-30° C. The mixture was stirred and concentrated under reduced pressure at a temperature below 30° C. 60 g of ethyl acetate was added to the concentrate. The mixture was concentrated under reduced pressure at a temperature below 30° C. The filter cake was rinsed with ethyl acetate, and dried under vacuum to give a light yellow solid with a yield of 79.9% and a purity of 87.6%.

In the above Example:

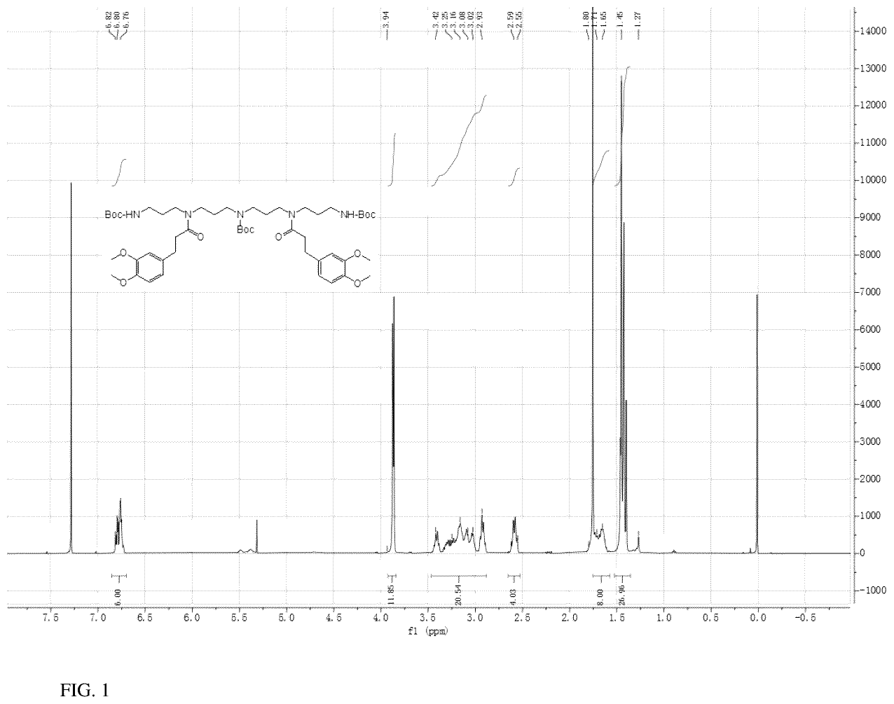

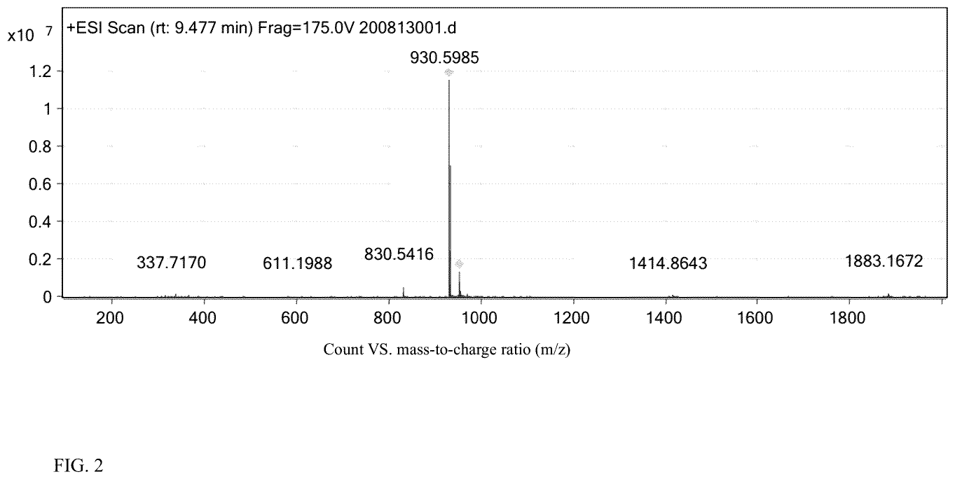

The results of nuclear magnetic resonance hydrogen spectrum and mass spectrum for Compound 2a are shown in FIGS. 1 and 2, respectively.

Conclusion: Compound 2a has a molecular formula of C₄₉H₇₉N₅O₁₂ with an average molecular weight of 930.19. In the ESI+mode high resolution mass spectrum of the test sample, there is a peak with m/z=930.5985, which is an M+H excimer ion peak. The data of high-resolution mass spectrum and nuclear magnetic resonance hydrogen spectrum show that the relative molecular mass detected for the test sample is consistent with the relative molecular mass of compound 2a, which matches the structures.

The above description is only for the purpose of illustrating the preferred examples of the present invention, and is not intended to limit the scope of the present invention. Any modifications, equivalents, and the like made without departing from the spirit and principle of the present invention shall fall in the protection scope of the present invention.

The foregoing examples and methods described herein may vary based on the abilities, experience, and preferences of those skilled in the art.

The certain order in which the steps of the method are listed in the present invention does not constitute any limitation on the order of the steps of the method.

1.-10. (canceled)

11. A compound having the following structure: 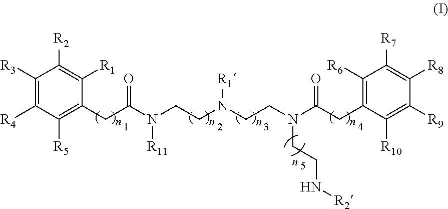
wherein,
R₁-R₁₀ are independently selected from: H, OH, alkoxy, aryloxy, and aralkoxy;
R₁₁ is R₃′ or 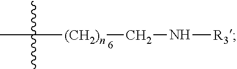
n₁-n₆ are independently selected from an integer of 0-10;
R₁′ is —X₁—R₁″, wherein X₁ is C(O)O or S(O)₂, and R₁″ is selected from: alkyl, alkenyl, aralkyl, and aryl;
R₂′ is —X₂—R₂″, wherein X₂ is C(O)O or S(O)₂, and R₂″ is selected from: alkyl, alkenyl, aralkyl, and aryl; and
R₃′ is —X₃—R₃″, wherein X₃ is C(O)O or S(O)₂, and R₃″ is selected from: alkyl, alkenyl, aralkyl, and aryl.

12. The compound according to claim 11 having the following structure:

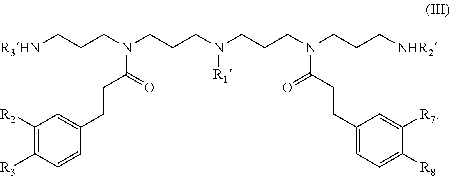

13. The compound according to claim 11, wherein R₂, R₃, R₇, and R₈ are independently selected from: H, OH, C₁-C₆ alkoxy, C₆-C₁₂ aryloxy, and C₇-C₁₂ aralkoxy.

14. The compound according to claim 11, wherein R₂, R₃, R₇, and R₈ are independently selected from: H, OH, and C₁-C₆ alkoxy.

15. The compound according to claim 11, wherein R₁″, R₂″, and R₃″ are independently selected from: C₁-C₆ alkyl, C₂-C₆ alkenyl, C₆-C₁₂ aryl, and C₇-C₁₂ aralkyl.

16. The compound according to claim 11, wherein R₁″, R₂″, and R₃″ are independently selected from: methyl, ethyl, tert-butyl, p-methylphenyl, allyl, and fluorenylmethyl.

17. The compound according to claim 11, wherein R₁′, R₂′, and R₃′ are independently selected from: tert-butoxycarbonyl, ethoxycarbonyl, methoxycarbonyl, allyloxycarbonyl, fluorenylmethoxycarbonyl, p-toluenesulfonyl, and mesyl.

18. A preparation method for the compound according to claim 11, wherein the compound is prepared from a compound of formula V having the following structure: 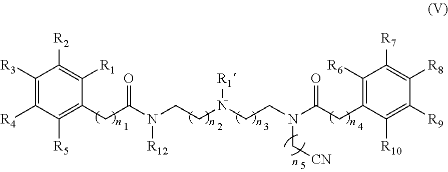
wherein R₁₂ is R₃′ or

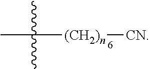

19. The preparation method according to claim 18, comprising mixing the compound of formula V with R₁₃-R₂′ and/or R₁₃-R₃′, alcohol, and a catalyst, and reacting, wherein R₁₃ is a leaving group.

20. The preparation method according to claim 19, wherein the alcohol is selected from: ethanol, isopropanol, or tert-butanol.

21. The preparation method according to claim 19, wherein the catalyst is Raney Ni or palladium on carbon.

22. The preparation method according to claim 19, wherein the reaction is performed at a temperature of 40-50° C.

23. The preparation method according to claim 19, wherein the reaction is performed under pressure of 1.0-2.0 MPa.

24. The preparation method according to claim 19, wherein the preparation method further comprises a purification step by column chromatography, wherein an eluent for the column chromatography is a mixture of acetone and n-heptane.

25. A preparation method for a polyamine derivative or a pharmaceutically acceptable salt thereof, comprising the following reaction scheme, wherein the polyamine derivative has a structure of formula VI: 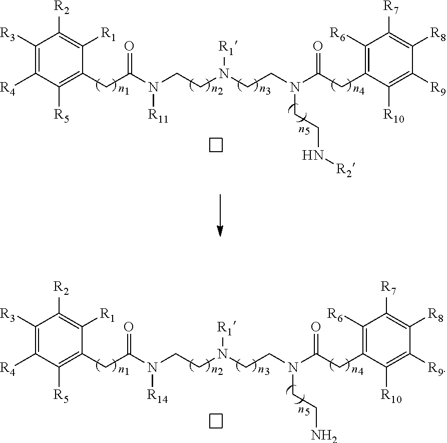
wherein,
R₁-R₁₀ are independently selected from: H, OH, alkoxy, aryloxy, and aralkoxy;
R₁₁ is R₃′ or 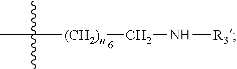
R₁₄ is H or 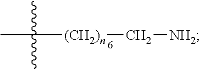
n₁-n₆ are independently selected from an integer of 0-10;
R₁′ is —X₁—R₁″, wherein X₁ is C(O)O or S(O)₂, and R₁″ is selected from: alkyl, alkenyl, aralkyl, and aryl;
R₂′ is —X₂—R₂″, wherein X₂ is C(O)O or S(O)₂, and R₂″ is selected from: alkyl, alkenyl, aralkyl, and aryl;
R₃′ is —X₃—R₃″, wherein X₃ is C(O)O or S(O)₂, and R₃″ is selected from: alkyl, alkenyl, aralkyl, and aryl; and
the step of preparing a compound of formula VI from a compound of formula I comprises mixing the compound of formula I with a solvent, slowly adding a removal reagent, and performing a reaction.

26. The preparation method according to claim 25, wherein the removal reagent is a solution of hydrogen chloride in an organic solvent.

27. The preparation method according to claim 26, wherein the organic solvent is selected from: ethyl acetate, cyclopentyl methyl ether, isopropyl acetate, methyl tert-butyl ether, methyl acetate, and propyl acetate.

28. The preparation method according to claim 25, wherein the reaction is performed at a temperature of 0-25° C.

29. The preparation method according to claim 25, wherein the preparation method further comprises a salt formation step by reacting the polyamine derivative with an acid; the salt formation step comprises slowly adding a solution of an acid in an organic solvent to a solution of the polyamine derivative and performing a reaction.

30. The preparation method according to claim 29, wherein the acid is selected from: hydrochloric acid, sulfuric acid, phosphoric acid, nitric acid, acetic acid, oxalic acid, malonic acid, succinic acid, benzoic acid, trifluoroacetic acid, maleic acid, fumaric acid, citric acid, tartaric acid, methanesulfonic acid, benzenesulfonic acid, and p-toluene sulfonic acid; and the step of slowly adding a solution of an acid in an organic solvent is performed at a temperature of 0-15° C.
